(19) United States
(12) Patent Application Publication
(10) Pub. No.: US 2005/0090659 A1
(43) Pub. Date: Apr. 28, 2005

(54) Phospholepid derivatives of nucleosides as antitumaorl medicaments

(76) Inventor: Dieter Herrmann, Heidelberg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/496,499

(22) PCT Filed: Nov. 18, 2002
(86) PCT No.: PCT/EP02/12908

(30) Foreign Application Priority Data
Nov. 21, 2001 (DE) 101-56-910.6
Mar. 4, 2002 (DE) 102-09-564.7

(51) Int. Cl. C07H 19/048; C07H 19/10

(52) U.S. Cl. 536/26.8

(57) ABSTRACT

The present invention relates to drugs containing phospholipid derivatives preferably of non-natural nucleosides corresponding to general formula I: in which 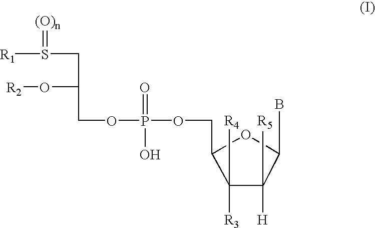
R₁ represents an alkyl chain with 10-14 carbon atoms,
R₂ represents an alkyl chain with 8-12 carbon atoms, n represents an integer equal to 0 to 2,
R₃ represents a hydroxy group,
R₄ and R₅ represent hydrogen, and B represents 5-fluorouracil, for use as antitumoral or antiproliferative active ingredients for the prophylaxis and/or curative, palliative or supportive treatment of tumor diseases or neoplasias, such as for example carcinomas, sarcomas, lymphomas or leukemias, both as therapeutic or prophylactic agents for monotherapy and in free or fixed combination with other modalities of prophylaxis or therapy.

The present invention relates to drugs containing phospholipid derivatives preferably of non-natural nucleosides corresponding to general formula I: 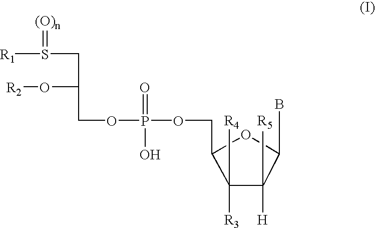
in which
R₁ represents an alkyl chain with 10-14 carbon atoms,
R₂ represents an alkyl chain with 8-12 carbon atoms,
n represents an integer equal to 0, 1 or 2,
R₃ represents a hydroxy group,
R₄ and R₅ represent hydrogen, and
B represents 5-fluorouracil,
   for use as antitumoral or antiproliferative active ingredients for the prophylaxis and/or curative, palliative or supportive treatment of tumor diseases or neoplasias, such as for example carcinomas, sarcomas, lymphomas or leukemias. The phospholipid derivatives corresponding to general formula I can also be provided in the form of their pharmacologically tolerable alkali or earth alkali salts.

Phospholipid derivatives of nucleosides are known from printed patent specification, EP 545 966 B1. The compounds are described to be substances with antiviral activity that are particularly suitable for the therapy and prophylaxis of infections caused by DNA viruses, such as for example the Herpes simplex virus, cytomegalovirus, Papovaviridae, Varicella zoster virus or Epstein-Barr virus, or RNA viruses, such as for example Togaviridae or, in particular retroviruses, such as for example HTLV-I and HTLV-II oncoviruses, as well as Lentiviridae, Visna and human immunodeficiency virus, HIV-1 and HIV-2. Moreover, the printed patent specification cited above emphasizes that compounds corresponding to general formula I are particularly suitable for the treatment of clinical manifestations of retroviral HIV infection in man, such as persistent generalized lymphadenopathy (PGL), advanced stage of AIDS-related complex (ARC), and the full clinical manifestation of AIDS. The compounds purportedly inhibit the proliferation of DNA or RNA viruses at the stage of virus-specific DNA or RNA transcription.

It is known from Proc. Natl. Acad. Sci. USA 83, 1911, 1986 and Nature 325, 773, 1987 that said substances can suppress the proliferation of retroviruses by inhibiting the enzyme, reverse transcriptase. Of special therapeutic interest in this context is the inhibiting effect of these compounds on HIV, the cause of immunodeficiency disease, AIDS. It is expressly pointed out in EP 545 966 that the antiviral or antiretroviral efficacy of these substances is not associated with cytotoxic effects at pharmacologically relevant dosages.

Lipid esters of nucleoside monophosphates with an antitumoral effect have already been described in WO 95/32984. The compounds according to the invention differ from the structures claimed therein by a changed substitution pattern at the C-2′ carbon atom of the sugar ring.

It was now surprisingly found that some of the phospholipid derivatives of nucleosides known from EP 545 966 have additional valuable pharmacological properties. These substances are particularly suitable for the prophylaxis and/or treatment of malignant tumors, such as for example malignomas, neoplasias, carcinomas, sarcomas or hematological tumor diseases, such as for example leukemias. Surprisingly, the compounds of the present invention act antitumoral or antiproliferative without exerting unspecific-toxic effects on other organ systems, such as for example bone marrow or gastrointestinal tract, at pharmacologically relevant dosages.

The compounds according to the invention corresponding to the following general formula, I: 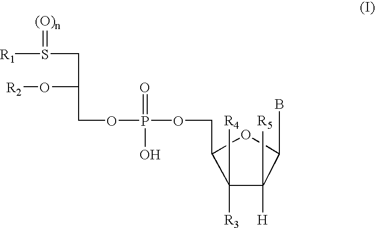
in which
R₁ represents an alkyl chain with 10-14 carbon atoms,
R₂ represents an alkyl chain with 8-12 carbon atoms,
n represents an integer equal to 0, 1 or 2,
R₃, R₄ and R₅ represent, independent of each other, hydrogen or a hydroxy group, provided that R₃ and R₄ are not both hydroxy groups, and
B represents a possibly modified or substituted nucleo-base as well as its physiologically tolerable salts of inorganic or organic acids including the various possible enantiomers, diastereomers or tautomers.

Preferably, the nucleo-base in general formula I represents cytosine, adenine, thymine, guanine, 5-fluorouracil, 5-bromouracil, 5-ethinyluracil, 5-propenyluracil, 5-trifluoromethyluracil, 2-amino-6-chloropurine, 2-chloroadenine, 2-fluoroadenine, 2,6-diaminopurine, 2-bromoadenine, 6-mercaptopurine or 6-methylmercaptopurine. Non-natural and, in particular, halogenated nucleo-bases are preferred. It is preferable for the purine bases to be connected to the sugar by means of the N₉ nitrogen, and for the pyrimidine bases to be connected by means of the N₁-nitrogen.

Preferred sugars comprise the following combinations of residues, R₃, R₄, and R₅:

| | R₃ | R₄ | R₅ |
|---|---|---|---|
| a) | OH | H | OH |
| b) | OH | H | H |
| c) | H | OH | H |
| d) | H | H | OH |

In general formula I, R₁ preferably represents a C₁₀-C₁₄ alkyl group with a linear chain. In particular, R₁ represents a decyl, undecyl, dodecyl, tridecyl or tetradecyl group. It is particularly preferred for R₁ to represent the undecyl and the dodecyl residue.

R₂ preferably represents a C₈-C₁₂ alkyl group with a linear chain, in particular an octyl, nonyl, decyl, undecyl or dodecyl group. It is particularly preferred for R₂ to represent the decyl and the undecyl residue.

Sulphur, being characterized by various oxidation states equal to 0, 1 or 2, represents a thioether, a sulfoxide or a sulfone. Thioethers and sulfoxides are particularly preferred.

The alkali and earth alkali salts are the preferred salts of the compounds corresponding to general formula I. Sodium, calcium, and magnesium salts are particularly preferred.

Particularly preferred are compounds corresponding to general formula I, in which R₅ represents hydrogen. These compounds are not yet known by name.

Particularly preferred is the compound, 5-fluoro-2′-deoxyuridine-5′-phosphoric acid-(3-dodecylmercapto-2-decyloxy)propyl ester as well as its sulfoxide and sulfone derivative (R₁ represents dodecyl, R₂ represents decyl, R₄/R₅ represent hydrogen, R₃ represents hydroxy, n equal to 0,1 or 2, and B represents 5-fluorouracil). These compounds have not been previously described in EP 545 966 or in WO 95/32984 and therefore are new.

An analogous route for the production of the compounds corresponding to general formula I is described in EP 0 545 966 B1 and WO 95/32984, the contents of which are incorporated herein by reference.

Compared to chemotherapeutic agents that have been used hitherto for the treatment of malignant neoplasias or tumors, the compounds according to the invention possess higher pharmacological-medical potency, improved efficacy and/or significantly lower toxicity, and therefore have a broader therapeutic range under in-vivo conditions. The compounds corresponding to general formula I are advantageous clinically-practically in that the administration of drugs containing these compounds can be maintained continuously for a longer period of time. Discontinuation or intermittent administration, as is often common or absolutely required with the cytostatic or chemotherapeutic agents currently used in the drug therapy of tumors due to their substantial undesired side effects, can be dispensed with in the application of drugs containing compounds corresponding to general formula I as antitumoral active ingredients. Only due to the good tolerability of the compounds corresponding to general formula I according to the invention, the continuous enteral or parenteral application of these substances is possible.

The compounds corresponding to general formula I contain asymmetrical carbon atoms, all optically active forms and racemic mixtures of the compounds are also an object of the present invention.

In this context, the diastereomers corresponding to general formulas IIa and IIb are of a particular interest: 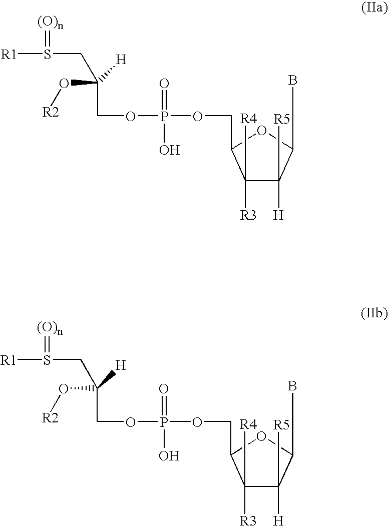
in which R₁, R₂, n, R₃, R₄, R₅, and B represent the same groups as in general formula I above, and can possibly be provided in the form of their salts.

Moreover, the tautomers of the compounds according to the invention and their physiologically tolerable salts of inorganic and organic acids or bases are also considered in the present invention. These also show selective antitumoral or antiproliferative properties.

Another object of the present invention are new substances corresponding to general formula I, in which
R₁ represents an alkyl chain with 10-14 C-atoms, and
R₂ represents an alkyl residue with 8-12 C-atoms,
n can equal 0, 1 or 2,
R₄ and R₅ represent hydrogen,
R₃ represents a hydroxy group, and
B represents the 5-fluorouracil residue,
   as well as their salts and all optically active forms and enantiomer mixtures.

Particularly preferred as new substances are compounds corresponding to general formula I, in which
R₁ represents a dodecyl residue, and
R₂ represents a decyl residue,
n can equal 0, 1 or 2,
R₄ and R₅ represent hydrogen,
R₃ represents a hydroxy group, and
B represents the 5-fluorouracil residue,
   as well as their salts and all optically active forms and enantiomer mixtures.

Compared to the compounds known hitherto, the new substances show antitumoral or antiproliferative effects at substantially lower dosages or have a substantially broader therapeutic spectrum under in-vitro or in-vivo conditions.

The compounds according to the present invention or their pharmaceutical preparations can also be used in free or fixed combination with other suitable drugs or active ingredients for the prophylaxis and/or curative, palliative or supportive treatment of tumor diseases or neoplasias.

Examples of these additional drugs include for example other cytostatic or chemotherapeutic agents in use for the prophylaxis and/or treatment of tumor diseases. This group includes for example nitrogen derivatives of mustard gas (e.g. cyclophosphamide, ifosfamide, trofosfamide, mafosfamide, chlorambucil, melphalan), aziridines and epoxides (e.g. thiotepa, triethylenemelamine, trenimone, treosulfane), alkyl-alkane sulfonates (e.g. busulfan), nitroso urea substances (e.g. carmustin, lomustin, semustin, nimustin, fotemustin, streptozotocin, chlorozotocin), mono-functional and non-classical alkylating agents (e.g. procarbazine, dacarbazine, hexamethylmelamine, mitozolomide, temozolamide, adozelesine and its derivatives), platinum derivatives (e.g. cisplatin, carboplatin, ormaplatin, oxaliplatin, tetraplatin, nedaplatin, CI-973, DWA 2114R, JM 216, JM 335, bis- and trans-platinum derivatives), folic acid antagonists or antifolate agents (e.g. methotrexate, trimetrexate, tomudex, edatrexate, lometrexol), purine and purine nucleoside analogs (e.g. 6-mercaptopurine, 6-thioguanine, pentostatine), pyrimidine and pyrimidine nucleoside analogs (e.g. 5-fluorouracil, 5-fluorouridine, 5-fluorodeoxyuridine, ftorafur, carmofur, tegafur, tegafur-gimestat-otastat, capecitabine, enocitabine, galocitabine, doxifluridine, cytosine arabinoside [Ara-C], azacitidine [Aza-C], Cl—F—AraA, peldesine, gemcitabine and its derivatives), anthracyclins and related intercalating compounds (e.g. doxorubicin and its morpholino derivatives, daunorubicin, epirubicin, idarubicin, pirarubicin, aclarubicin, amrubicin, MX-2, mitoxantrone, losoxantrone, amsacrine, and pyrazoloacridine), antibiotic cytostatic or chemotherapeutic agents (e.g. bleomycins, peplomycin, mitomycin C, actinomycin D, mithramycin, clecarmycin, FK-317), microtubule inhibitors such as for example vinca alkaloids (e.g. vincristine[sulfate], vinblastine[sulfate], vindesine[sulfate], vinorelbine), AM-132, KW-2170, rhizoxine, palmitoyl rhizoxine, dolostatins (e.g. dolostatin 10), phomopsins, halichondrins, homohalichondrins, spongistatins, combrestatins, steganacine, taxanes (e.g. paclitaxel, docetaxel, baccatin III and their derivatives), topoisomerase inhibitors, such as for example epipodophyllotoxins (e.g. etoposide, etoposide phosphate, teniposide), J 1070088, TOP-53 or camptothecine and its analogs (e.g. 9-amino-camptothecine, topotecane, irinotecane, exatecane, CPT-11), L-asparaginase, sparfosate, hydroxyurea, mitotane, epothilone and deoxyepothilone as well as their derivatives, fludarabine, fludarabine phosphate, 2-chlorodeoxyadenosine, 2′-deoxycoformycin, homoharringtonine, sumarin, antitumoral-immunosuppressive-acting drugs, such as for example cyclosporine, rapamycine, deoxyspergualines and corticoids (e.g. cortisol, cortisone, prednisone, prednisolone, para-, β-, dexamethasone).

The compounds of the present invention and their pharmaceutical preparations can also be used in free or fixed combination with tyrosine kinase inhibitors (e.g. SU-5416, KT-8391, KT-5555), farnesyltransferase inhibitors (e.g. BMS-214662, ER-51785, R 115777), thymidylate synthase inhibitors (e.g. 2′-deoxy-2′-fluoro-4′-thioarabinosylcytosine, raltitrexed, TK-117, TAS 102, TAS 103), DNA polymerase inhibitors (e.g. 1-(2-deoxy-2-methylene-D-erythro-pentofuranosyl)cytosine [DMDC, Y-26436], CS-682), histone deacylase inhibitors (e.g. MS-275), metalloproteinase inhibitors (e.g. marimastat, batimastat, CGS-27023A, MMI-166, S-3304), P-glycoprotein inhibitors (e.g. valspodar, MS-209, PAK-104P, LY-335979), cyclooxygenase-2 inhibitors (e.g. R-109339), inhibitors of phosphatase, adenosine deaminase, RNA polymerase, protein kinase C (e.g. hexadecylphosphocholine, calphostin, gossipol, quercetin, fisetin, staurosporins [e.g. midostaurin, 7-hydroxystaurosporin, KW-2401]), antiangiogenesis agents and inhibitors of angiogenesis (e.g. FMPA, TNP-470, Anti-VEGF/VPF monoclonal antibody) or with agonists/inductors of apoptosis (e.g. AOP 99.0001, irofulvene, NCO-700, T 215, TAC-101) for the prophylaxis and/or treatment of tumor diseases or neoplasias.

Moreover, the compounds corresponding to general formula I according to the invention can also be used for the prophylaxis and/or treatment of tumor diseases or neoplasias in free or fixed combination with hormones or antihormones that are in common use for prophylaxis and/or therapy in oncology. This includes for example androgens, estrogens, gestagens, antiandrogens, antiestrogens, and antigestagens as well as inhibitors of releasing hormones, such as for example LHRH (luteinizing hormone-releasing hormone), their analogs, antagonists, and superagonist. Examples of the latter compounds include busereline(acetate), gosereline(acetate), leuproreline(acetate), triptoreline(acetate). Examples of LHRH antagonists are antide, ramorelix, cetrorelix, teverelix, abarelix, and ORG 30850.

Examples of hormone agonists that can be combined with the compounds according to the invention include for example the estrogen derivatives, fosfestrol, chlorotrianisen, ethinylestradiol, diethylstilbestrol, polyestradiolphosphate, and the gestagen analogs, medroxyprogesterone acetate, megestrol acetate and fluoxymesterone.

The compounds corresponding to formula I according to the invention can also be used for the prophylaxis and/or treatment of tumor diseases or neoplasias in free or fixed combination with 5α-reductase inhibitors (e.g. epristeride, finasteride, turosteride, LV 654066), steroidal and non-steroidal antiandrogens (e.g. cyproterone acetate, flutamide, BMOT, anandrone [RU 23908], faslodex, casodex [ICI 176334], WIN 49596), non-steroidal antiestrogens (e.g. tamoxifen, diethylstilbestrol, clomiphene, nafoxidine, MER-25, droloxifene, toremifene, zindoxifene, tetramethyl-HES, LY 117018) and jointly with antiestrogens such as for example ICI 164384, ZK 119010, ICI 182780, RU 58668. Examples of antigestagenic combination partners are mifepristone (RU 486) and onapristone (ZK 98.299).

Other suitable combination partners for the compounds of the invention are aromatase inhibitors, such as for example aminoglutethimide, rogletimide, letrozole, as well as steroidal aromatase inhibitors such as for example exemestan, formestan, minamestan, atamestan, MDL 18962, ORG 30958, and non-steroidal aromatase inhibitors, such as for example fadrozol, vorozol, anastrozol, CGS-20267.

The compounds corresponding to formula I according to the invention can also be used in free or fixed combination with uracil, eniluracil, 3′-ethinyluridine, 3′-ethinylcytidine, fluoropyrimidines (e.g. (E)-2′-deoxy-2′-(fluoromethylene)cytidine, MDL-1 01731) and/or dihydropyrimidine dehydrogenase (DPD) inhibitors, for the prophylaxis and/or treatment of tumor diseases or neoplasias such as for example colorectal, mammary, ovarian, prostatic, pancreatic or lung carcinoma.

Particularly the following fluoropyrimidines or fluoropyrimidines formulations are suitable combination partners, in free or fixed combination, of the compounds according to the invention:
UFT, a combination of uracil and tegafur (1-[2-tetrahydrofuranyl]-5-fluorouracil) at a fixed molar ratio of 4:1;
S-1 (BMS 247617), a combination of tegafur and two 5-fluorouracil modulators, namely CDHP (chloro-2,4-dihydroxypyrimidine, a potent DPD inhibitor) and potassium oxonate,
BOF-A2 (emitefur), a drug consisting of 1-ethoxymethyl-5-fluorouracil (EM-FU) and 3-cyano-2,6-dihydroxypyridine (CNDP), a potent DPD inhibitor,
Eniluracil (5-ethinyl-2,4(1H,3H)-pyrimidinedione), a potent and irreversible DPD inhibitor.
Tegafur (1[2-tetrahydrofuranyl]-5-fluorouracil)
Capecitabine, enocitabine or galocitabine.

By combining the compounds corresponding to formula I according to the invention with uracil, eniluracil, 3′-ethinyluridine, 3′-ethinylcytidine, fluoropyrimidines or DPD inhibitors or modulators, such as for example UFT, CDHP, CNDP, etc., another therapeutic advantage is attained in that the antitumoral potency, tolerability, and stability of the compounds according to the invention is significantly increased due to the inhibition of DPD.

The compounds corresponding to formula I according to the invention can also be used for the prophylaxis and/or therapy of tumor diseases or neoplasias in free or fixed combination with cytokines or cytokine receptor agonists or antagonists. Cytokine combination partners include for example interleukins (e.g. interleukins [IL] 1-18 [edodekin], in particular IL 1, 2, 3, 6, 10, 11, 12), interferons (e.g. interferon α,β,γ), tumor necrosis factors (e.g. TNF α,β) , TNF agonists (e.g. sonermin) as well as transforming growth factors (e.g. TGF α, β).

Also suitable for combination therapy with the compounds according to the invention are hematopoietic growth factors. Pertinent examples include for example erythropoietin, thrombopoietin, granulocyte colony-stimulating factor (G-CSF), granulocyte-macrophage colony-stimulating factor (GM-CSF), and macrophage colony-stimulating factor (M-CSF).

Due to their high antitumoral potency at very good tolerability, the compounds corresponding to formula I according to the invention are suitable for use in combination with specific or unspecific, active or humoral or cellular passive modalities of immunotherapy, for the prophylaxis and/or treatment of tumor diseases and neoplasias.

Examples of specific active immunotherapies include for example the injection or application of irradiated tumor cells or tumor-associated antigens or immunization with genetically modified tumor cells, e.g. with cytokine gene transfectants, or with virus-infected tumor cells. In this context, unspecific active immunotherapies comprise for example the application of immunostimulating or -modulating substances, such as for example BCG, iscador, Ok-432, levamisol, ubenimex, lentinam, bestatin, MER, MTP—PE.

Passive humoral immunotherapies, in which the compounds corresponding to formula I according to the invention can be used for the prophylaxis and/or treatment of tumor diseases and neoplasias include for example the injection or application of murine, human or humanized monoclonal antibodies or immuno-conjugates, e.g. radioisotope-, cytostatic agent- or toxin-coupled (immunotoxins) monoclonal antibodies (e.g. gentuzumab, edrecolomab, trastuzumab, rituximab, lintuzumab, ACA-11, V-10500, Anti-HM1.24 MAB, C225). Further examples of passive humoral immunotherapies include genetically-modified monoclonal antibodies, bispecific antibodies or immunoglobulin-T-cell receptor chimeras. The compounds corresponding to formula I according to the invention can also be used for the prophylaxis and/or treatment of tumor diseases and neoplasias in combination with passive cellular immunotherapies. Examples of this type of therapeutic modality include for example adoptive immunotherapies with cytotoxic effector cells, such as for example lymphokine-activated killer cells (LAK), adherent LAK, large granular lymphocytes (LGL), natural killer cells (NK), tumor-infiltrating lymphocytes (TIL), dendritic cells or cytotoxic T-lymphocytes (CTL) as well as the transfer of genetically-modified effector cells (gene therapy, e.g. adenoviral-p53).

The compounds corresponding to formula I according to the invention show valuable pharmacological properties also when combined with radiotherapy. Due to their high antitumoral potency, the combination with radiotherapy for the treatment of tumor diseases and neoplasias produces synergistic antitumoral and antiproliferative effects. On the other hand, the known unspecific-cytotoxic side effects of radiotherapy on rapidly proliferating cells, such as for example bone marrow cells or mucosal cells of the gastrointestinal tract, are not increased in combinations of compounds corresponding to formula I according to the invention and radiotherapy which is due to the excellent organ/tissue tolerability of these substances, and therefore the therapeutic range of combination therapy is broadened substantially.

The drugs according to the invention containing compounds corresponding to formula I according to the invention for the prophylaxis and/or treatment of tumor diseases and neoplasias can be applied in liquid or solid form by an enteral or parenteral route. The common application forms are suitable, such as for example tablets, capsules, coated tablets, syrups, solutions, sprays or suspensions. It is preferable to use as an injection medium water containing the usual additives of solutions for injections, such as stabilization agents, solutions mediators, and buffers. Additives of this type include for example tartrate and citrate buffer, ethanol, complexing agents, such as ethylenediamine tetraacetic acid and its non-toxic salts, high molecular polymers, such as liquid polyethylene oxide to regulate the viscosity. Liquid carrier substances for solutions for injection must be sterile and are preferably filled into vials. Solid carrier substances include for example starch, lactose, mannitol, methylcellulose, talcum, highly disperse silica acids, higher molecular fatty acids, such as for example stearic acid, gelatine, agar-agar, calcium phosphate, magnesium stearate, animal and plant fats, solid high molecular polymers, such as polyethylene glycols, etc. Suitable preparations for oral application can contain flavoring or sweetening agents, if desired.

The dosage can depend on a variety of factors, such as type of application, species, age or individual status. The compounds according to the invention are usually applied at dosages of 0.1-100 mg per kg body weight per day, preferably 0.2-80 mg per kg body weight per day. It is preferably to distribute the daily dose over 2-5 applications with each application involving the administration of 1-2 tablets or vials with an active ingredient content of 0.5-500 mg. The tablets can also be provided in the form of a delayed release preparation which reduces the number of daily application to 1-3. The active ingredient content of the delayed release tablets can be 2-1000 mg. The active ingredient can also be administered by means of 1-3 parenteral applications or by permanent infusion, whereby quantities of 5-1000 mg per day are usually sufficient.

Combining compounds corresponding to formula I with one or several additional active ingredients, the active ingredients can be provided either in a fixed combination in the same form of administration, e.g. tablet or vial, or in one or several other forms of administration. The latter is required, for example, if the active ingredients to be combined are not compatible with each other, e.g. due to reactions during storage. It is self-evident that with regard to the combination of three or more active ingredients, these can all be manufactured as a fixed combination in one form of administration or in two or more forms of administration to be applied in free combination.

The following examples are to illustrate the invention, though without restricting the scope of the invention.

### EXAMPLE 1

### Antitumor activity of 5-fluoro-2′-deoxyuridine-5′-phosphoric acid-(3-dodecyl-thio-2-decyloxy)propylester (substance A) and 3′-azido-3′-deoxythymidine-5′-phosphoric acid-(3-dodecylthio-2-decyloxy)propylester (substance B) in the MethA fibrosarcoma model

The substances, 5-fluoro-2′-deoxyuridine-5′-phosphoric acid-(3-dodecyl-thio-2-decyloxy)propylester (substance A) and 3′-azido-3′-deoxythymidine-5′-phosphoric acid-(3-dodecylthio-2-decyloxy)propylester (substance B), have been tested in the murine MethA fibrosarcoma model, amongst other tests, under in-vivo conditions for their antitumoral or antiproliferative potency and efficacy.

MethA fibrosarcoma cells were propagated intraperitoneal (i.p.) in the form of ascites tumor in female CB6F₁ mice (Charles River Laboratories, Sulzfeld, Germany). For the entire duration of the tests, the animals were kept in macrolon cages under laminar flow conditions at 23±1° C. room temperature, 55±15% relative humidity and a light-dark cycle of 12 h each. The mice were fed a standard diet (Ssniff-Spezialdiäten GmbH, Soest/Westfalen, Germany) and had access to water ad libidum. Prior to enrolment in the corresponding experiment, the animals were accustomed to the conditions for at least 14 days. The animals were routinely checked for infection by murine viruses.

To test the active substance, female CB6F₁ mice 6-8 weeks of age were inoculated with 1×10⁵ MethA fibrosarcoma cells per mouse by the subcutaneous (s.c.) route. The tumor growth in control group mice and in animals of the verum-treated groups was determined regularly in weekly intervals by measuring the two perpendicular tumor diameters as described in Hermann D. B. J., Pahlke W., Opitz H.-G. and Bicker U., Cancer Treatment Reviews, 17, 247-252, 1990. The test substances were tested in a dose-dependent fashion with once weekly i.p. administration in phosphate-buffered saline (PBS). Animals in the control group were treated with placebo (PBS).

Table 1 shows the effect of substances A and B on tumor growth in the MethA fibrosarcoma model under in-vivo conditions. Tumor volumes on days 21 and 28 after tumor cell inoculation are expressed as medians of 10 animals per experimental group.

TABLE 1
Antitumor activity in the MethA fibrosarcoma model

| | | Dose | | |
|---|---|---|---|---|
| | | (mg/kg/ | Tumor volume (mm³)^{a} | |
| Group | Substance | application) | Day 21 | Day 28 |
| 1 | Control | — | 4.032 (—) | 9.827 (—) |
| | (Placebo^{b}) | | | |
| 2 | Substance A | 3 | 1.649* (59.1) | 4.163* (57.6) |
| 3 | Substance A | 30 | 416** (89.7) | 1.254** (87.2) |
| 4 | Substance A | 100 | 357** (91.1) | 762** (92.2) |
| 2 | Substance B | 3 | 3.998 (0.8) | 10.228 (+4.1) |
| 3 | Substance B | 30 | 4.102 (+1.7) | 9.963 (+1.4) |
| 4 | Substance B | 100 | 4.021 (0.3) | 10.098 (+2.8) |

^{a}Median; 10 animals per group; percent inhibition relative to the control values of group 1 in parentheses (+ means increase)
^{b}Placebo: Phosphate-buffered saline (PBS)
*p ≦ 0.05,
**p ≦ 0.01; Mann-Whitney test

The results indicate that the substance A according to the invention surprisingly inhibits tumor growth highly significantly and in a dose- and time-dependent fashion, i.e. acts antitumoral and antiproliferative.

Substance B (Example 1a of EP 545966) shows no antitumoral or antiproliferative properties.

### EXAMPLE 2

### Tolerability of 5-fluoro-2′-deoxyuridine-5′-phosphoric acid-(3-dodecyl-thio-2-decyloxy)propylester (substance A) under in-vivo conditions

Substance A was tested in female NMRI mice for its tolerability. The animals were kept under the same conditions as described in Example 1.

Female NMRI mice, 6-8 weeks of age (Charles River Laboratories, Sulzfeld, Germany), were treated with 1 or 1.5 g/kg of substance A administered by an esophageal probe. Subsequently, the following toxicity parameters were determined:
Blood counts, including white blood cell concentration (WBC), red blood cells concentration (RBC), hemoglobin (HB), hematocrit (HCT), platelet concentration (PLT), mean corpuscular volume (MCV), mean corpuscular hemoglobin (MCH) and mean corpuscular hemoglobin concentration (MCHC),
Bone marrow cell count, i.e. number of bone marrow cells per femur (M/femur),
Body weight
Organ weights, including weight of colon, heart, brain, intestines, lungs, liver, stomach, spleen, kidneys, ovaries.

The results of these experiments are shown in Table 2.

TABLE 2
Tolerability of 5-fluoro-2′-deoxyuridine-5′-
phosphoric acid-(3-dodecyl-thio-2-decyloxy)propylester
(substance A) under in-vivo conditions^{a}

| | Control | Substance A | Substance A |
|---|---|---|---|
| Parameter | (Placebo^{b}) | (1 g/kg) | (1.5 g/kg) |
| Bone marrow cell | 28.28 ± 0.99 | 28.48 ± 0.86 | 31.44 ± 1.51 |
| count | | | |
| (M/Femur) | | | |
| Blood levels: | | | |
| WBC (k/μl) | 12.20 ± 0.66 | 14.34 ± 2.18 | 16.97 ± 6.05 |
| RBC (M/μl) | 9.24 ± 0.17 | 9.31 ± 0.23 | 8.34 ± 0.56 |
| HB (g/l) | 17.16 ± 0.31 | 17.85 ± 0.39 | 15.79 ± 0.92 |
| HCT (%) | 46.54 ± 0.97 | 47.93 ± 1.16 | 43.42 ± 2.77 |
| MCV (fl) | 50.32 ± 0.51 | 51.50 ± 0.58 | 52.09 ± 0.81 |
| MCH (pg) | 18.62 ± 0.14 | 19.28 ± 0.22 | 19.09 ± 0.37 |
| MCHC (g/dl) | 36.91 ± 0.36 | 37.29 ± 0.44 | 36.53 ± 0.38 |
| PLT (k/μl) | 1115 ± 48 | 1153 ± 41 | 1431 ± 172 |
| Body weight (g) | 29.7^{c} | 28.7^{c} | 27.9^{c} |
| Organ weight: | | | |
| Colon (g) | 429 ± 17 | 373 ± 15 | 392 ± 29 |
| Heart (g) | 130 ± 3 | 133 ± 4 | 126 ± 9 |
| Lungs (g) | 218 ± 5 | 226 ± 9 | 206 ± 9 |
| Liver (g) | 1.597 ± 53 | 1.602 ± 42 | 1.739 ± 81 |
| Kidney (g) | 380 ± 9 | 361 ± 17 | 351 ± 26 |
| Spleen (g) | 131 ± 12 | 125 ± 9 | 204 ± 26 |
| Stomach (g) | 226 ± 6 | 232 ± 7 | 232 ± 16 |
| Intestines (g) | 1.494 ± 73 | 1.622 ± 61 | 1.807 ± 112 |
| Brain (g) | 372 ± 21 | 399 ± 8 | 387 ± 18 |
| Ovaries (g) | 197 ± 18 | 183 ± 17 | 203 ± 33 |

^{a}Mean ± SEM; 10 animals per group
^{b}Placebo: Phosphate-buffered saline (PBS)
^{c}Median

This data shows that even at very high dosages, i.e. 1 or 1.5 g/kg of body weight, substance A causes no significant reduction in the tolerability parameters listed above as compared to the placebo (potable water)-treated control group. These results demonstrate that substance A has no unspecific-toxic properties under in-vivo conditions even at very high dosage. Even at very high dosage of substance A, there is no evidence of bone marrow suppression, hematotoxicity or unspecific-toxic organ intolerance.

In summary, the results of Examples 1 and 2 show that the compounds corresponding to general formula I according to the invention surprisingly have very good antitumoral or antiproliferative efficacy under in-vivo conditions, but no unspecific-toxic properties, such as bone marrow suppression, hematotoxicity or organ toxicities. Other compounds described in EP 545 966, whose structure does not correspond to formula I, do not show these pharmacological properties.

### EXAMPLE 3

### Production of 5-fluoro-2′-deoxyuridine-5′-phosphoric acid-(3-dodecyl-thio-2-decyloxy)propylester

As described in WO 95/32984, 52 g of crude rac-(3-dodecylthio-2-decyloxy)propyl-dihydrogenphosphate and 53.3 g of 2,4,6-triisopropylbenzenesulfochloride were stirred in 600 ml abs. pyridine under Argon for one hour at room temperature. Then 27.4 g of 3′-acetyl-2′-deoxy-5-fluorouridine were added and the mixture was stirred for another 16 h.

Subsequently, 100 ml of water were added and the suspension was stirred for 10 min. The solvent was removed in a vacuum, the residue was redistilled twice with 200 ml toluene each, and the residual viscous oil was suspended in 700 ml MTBE (methyl-tertiary-butylether). After heating to 40° C., sonication in an ultrasound bath, and cooling to 20° C., the precipitate was filtered off, and washed with 100 ml of MTBE.

The filtrate was extracted three times with 150 ml of 2 N hydrochloric acid, the organic phase was evaporated, and the residue dissolved in 400 ml of methanol. After adding 42 ml of 30% sodium methylate solution (pH=11), briefly stirring, and adding 5 ml of glacial acetic acid, the low-boiling substances were distilled off under a vacuum.

The residue was dissolved in 700 ml of MTBE and extracted twice with 100 ml 2 N hydrochloric acid each. The organic phase was evaporated and it was redistilled with 100 ml of toluene.

The residue contained the compound identified in the title in the form of the free acid.

### EXAMPLE 4

### Production of 5-fluoro-2′-deoxyuridine-5′-phosphoric acid-(3-dodecyl-thio-2-decyloxy)propylester, calcium salt

The crude product of the previous reaction was dissolved in 1 l of acetone at 50° C. The corresponding calcium salt was precipitated by slowly adding drops of 30 g of calcium acetate in 75 ml of water under stirring and cooling to room temperature over the course of 1 h.

The precipitate was aspirated, washed with acetone, and dried in a vacuum. Yield: 106 g of the crude calcium salt.

### EXAMPLE 5

### Chromatographic purification of 5-fluoro-2′-deoxyuridine-5′-phosphoric acid-(3-dodecyl-thio-2-decyloxy)propylester

The calcium salt obtained in the previous reaction was suspended in 600 ml of MTBE and 200 ml of 2 N hydrochloric acid. The organic phase was then separated and evaporated in a vacuum. Yield: 67.4 g. The crude product was dissolved in 140 ml of methanol at 40° C., and 36 ml of triethylamine and 20 ml of water were added.

Aliquots of the product were purified by preparative HPLC on LiChroprep RP18, 25-40 μm (column: Ø50 mm, length 200 mm) using methanol/0.04 M sodium acetate solution (80/20) as the elution agent.

The fractions containing the product were then combined and their volume was reduced to 30% of the original volume in a vacuum. Under stirring, 20 g of calcium acetate in 40 ml of water were added in the form of individual drops and the suspension was stirred for another 1 h. The precipitate was aspirated and dried in a vacuum. Yield: 42.2 g of the calcium salt.

### EXAMPLE 6

### Production of 5-fluoro-2′-deoxyuridine-5′-phosphoric acid-(3-dodecyl-thio-2-decyloxy)propylester, sodium salt

A total of 42.2 of the calcium salt were suspended in 400 ml of MTBE and 200 ml 2 N hydrochloric acid. The organic phase was separated, filtered through kieselguhr, and then evaporated in a vacuum. The residue was redistilled twice with 100 ml of toluene each, and then dissolved in 80 ml of toluene at 40° C. The pH was adjusted to a value of 7 by adding 30% sodium methylate solution under stirring, and then the solution was added in the form of individual drops to 1.4 l of acetone at 50° C.

After stirring for 1 h, the precipitate was aspirated, washed with acetone, and dried in a vacuum. Yield: 41.2 g, Fp: 175° C. (disintegration).

### EXAMPLE 7

Production of 5-fluoro-2′-deoxyuridine-5′-phosphoric acid-[(2R)(3-dodecyl-thio-2-decyloxy)]propylester

Following the procedures of Examples 3 to 6, it was possible to start from (R)-(3-dodecylthio-2-decyloxy)propyl-dihydrogenphosphate to produce the conjugate, 5-fluoro-2′-deoxyuridine-5′-phosphoric acid-[(2R)(3-dodecyl-thio-2-decyloxy)]propylester, in the form of the free acid, calcium salt, and sodium salt.

The identity of the substance was confirmed by thin layer chromatography by reference to authentic samples.

### EXAMPLE 8

### Production of 5-fluoro-2′-deoxyuridine-5′-phosphoric acid-[(2S)(3-dodecyl-thio-2-decyloxy)]propylester

Following the procedures of Examples 3 to 6, it was possible to start from (S)-(3-dodecylthio-2-decyloxy)propyl-dihydrogenphosphate to produce the conjugate, 5-fluoro-2′-deoxyuridine-5′-phosphoric acid-[(2S)(3-dodecyl-thio-2-decyloxy)]propylester, in the form of the free acid, calcium salt, and sodium salt.

The identity of the substance was confirmed by thin layer chromatography by reference to authentic samples.

Enantiomerically pure (R)-(3-dodecylthio-2-decyloxy)propyl-dihydrogenphosphate or (S)-(3-dodecylthio-2-decyloxy)propyl-dihydrogenphosphate was produced by separation of the racemate by means of diastereomeric salts.

### EXAMPLE 9

### Combination of the sodium salt of 5-fluoro-2′-deoxyuridine-5′-phosphoric acid-[2-(3-dodecyl-thio-2-decyloxy]propylester with cisplatin, doxorubicin, vincristine, and camptothecin

The effect of the combination containing substance A and cisplatin, doxorubicin, vincristine, and camptothecin, was tested in proliferation experiments. For this purpose, substance A was dissolved in medium at a stock concentration of 1 mg/ml. The other substances were dissolved in water or DMSO (dimethylsulfoxide) at the same stock concentration of 1 mg/ml. The series' of experiments were performed in 96-well plates. For each titration series, either 75 μl of the solution of the substance were placed in the first well and then 25 μl each were transferred to the next row or 100 μl were placed in the first well and 50 μl each were transferred. Then 50 μl cell suspension (5×10⁴ cells/ml, K562 cells, RPMI 1640 medium) each were added and the plates were incubated for 24 to 78 hours at 37° C., 5% CO₂ and 95% humidity. Cells in the absence of substance and pure medium served as the controls.

The in-vitro activity of the test substances was determined by colorimetry on the basis of cleavage of the tetrazolium salt, WST-1 (Roche Molecular Biochemicals, Mannheim, Del.). For this purpose, the cultures were incubated with 10 μl WST for 4 hours. Then the plates were shaken gently. The optical density was measured with an ELISA reader (Spectra MAX 340_{PC}, Molecular Devices, Ismaning, Del.) at wavelengths from 440 to 650 nm.

The IC₅₀ value was determined for each substance. In the case of the combination, one of the substances was used at a concentration just short of showing an antiproliferative effect and the IC₅₀ value of the combination was then determined after the addition of the second substance.

The combination with cisplatin resulted in a reduction of the IC₅₀ value of substance A and of cisplatin by 25% and 50%, respectively.

The combination with doxorubicin resulted in a reduction of the IC₅₀ value of substance A and of doxorubicin by approx. 30% and approx. 50%, respectively.

The combination with vincristine resulted in a reduction of the IC₅₀ value of substance A and of vincristine by approx. 60% and approx. 65%, respectively.

The combination with camptothecin resulted in a reduction of the IC₅₀ value of substance A and of camptothecin by approx. 40% and approx. 90%, respectively.

It is evident from these tests that a synergistic enhancement of the efficacy can be attained by combining these substances.

### EXAMPLE 10

### (5-fluorouridine)-5′-phosphoric acid-(3-dodecylsulfinyl-2-decyloxy)propylester

A total of 5 g of (5-fluorouridine)-5′-phosphoric acid-(3-dodecylthio-2-decyloxy)propylester were suspended in 50 ml of glacial acetic acid, then 5 ml of 30% hydrogen peroxide were added and the mixture was stirred for 4 hours at room temperature. Then the solvent was removed in a rotary evaporator and the residue was purified by preparative column chromatography on RP 18 using methanol/0.1 M acetate buffer as the elution agent.

The fractions containing the product were evaporated, the residue was stirred in acetone, and the precipitate was aspirated. After drying in a vacuum drying cabinet at 40° C., a total of 4.5 g of the sulfoxide were isolated.

Melting point: 214-216° C. (disintegration), Rf=0.27 (BuOAc/iPrOH/H₂O/NH₄OH 3/5/1/1),

³¹ P-NMR: δ=0.027 ppm.

### EXAMPLE 11

### (5-fluorouridine)-5′-phosphoric acid-(3-dodecylsulfonyl-2-decyloxy)propylester

A total of 10 g (5-fluorouridine)-5′-phosphoric acid-(3-dodecylthio-2-decyloxy)propylester were suspended in 100 ml of glacial acetic acid, then 25 ml of 30% hydrogen peroxide were added and the mixture was stirred for 6 hours at 50° C. Then another 13 ml of hydrogen peroxide were added and the mixture was stirred for another 7 hours.

Subsequently, the solvent was removed in a rotary evaporator and the residue was purified by preparative column chromatography on RP 18 using methanol/0.1 M acetate buffer as the elution agent.

The fractions containing the product were evaporated, the residue was stirred in acetone, and the precipitate was aspirated. After drying in a vacuum drying cabinet at 40° C., a total of 8.5 g of the sulfoxide were isolated.

Melting point: 204-207° C. (disintegration), Rf=0.29 (BuOAc/iPrOH/H₂O/NH₄OH 3/5/1/1),

³¹P-NMR: δ=0.073 ppm

### EXAMPLE 12

### Tablet Formulation

1.50 kg 5-fluoro-2′-deoxyuridine-5′-phosphoric acid-(3-dodecyl-thio-2-decyloxy)propylester, calcium salt

1.42 kg microcrystalline cellulose

1.84 kg lactose

0.04 kg polyvinylpyrrolidone

0.20 kg magnesium stearate

are mixed as the dry substances, then wet-granulated with water, dried, and pressed into tablets with a weight of 500 mg using a rotary tablet press.

### EXAMPLE 13

### Solution for Injection

10.0 g of 5-fluoro-2′-deoxyuridine-5′-phosphoric acid-(3-dodecyl-thio-2-decyloxy)propylester, sodium salt, are dissolved in 500 ml of saline and then filled into vials of 5 ml each and sterilized. The solution can be applied by intravenous injection.

1-28. (canceled)

29. Compounds corresponding to general formula I: 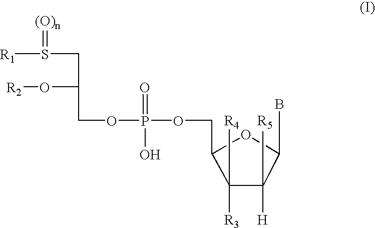
in which
R1 represents dodecyl,
R2 represents decyl,
n represents an integer equal to 0, 1 or 2,
R3 represents a hydroxy group,
R4 and Rs represent hydrogen or, and
B represents 5-fluorouracil,
their physiologically tolerable salts, stereoisomers or tautomers as well as all optically active forms and enantiomer mixtures.

30. 5-Fluoro-2′-deoxyuridine-5′-phosphoric acid-(3-dodecyl-thio-2-decyloxy)-propylester and its salts, stereoisomers or tautomers as well as all optically active forms and enantiomer mixtures.

31. Compounds corresponding to general formula IIa or IIb, 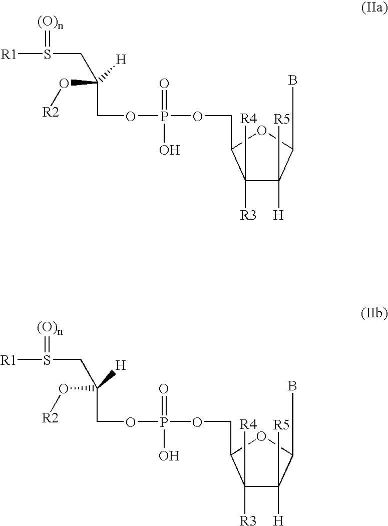
in which
R1 represents a dodecyl residue and
R2 represents a decyl residue,
n can be equal to 0, 1 or 2,
R4 and R5 represent hydrogen,
R3 represents a hydroxy group, and
B represents the 5-fluorouracil residue, as well as the salts thereof.

32. Use of a compound according to claim 29 for the prophylaxis and/or curative, palliative or supportive treatment of tumor diseases or neoplasias.

33. Use according to claim 32, whereby the tumor disease or neoplasia is a carcinoma, in particular a colorectal, mammary, ovarian, prostatic, lung or pancreatic carcinoma, sarcoma, lymphoma or a leukemia.

34. Drugs for the prophylaxis and/or therapy of tumor diseases or hematological neoplasias containing compounds corresponding to general formula I, 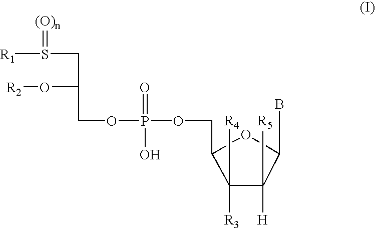
in which
R1 represents dodecyl,
R2 represents decyl,
n represents an integer equal to 0, 1 or 2,
R3 represents a hydroxy group,
R4 and Rs represent hydrogen, and
B represents 5-fluorouracil,
or their physiologically tolerable salts, stereoisomers or tautomers as well as optically active forms and enantiomer mixtures as active ingredients as well as liquid and/or solid carrier substances.

35. Drugs according to claims 34, whereby n is equal to 0 or 1.

36. Drugs according to claim 36, characterized in that it contains one or several additional active ingredients for the prophylaxis and/or curative, palliative or supportive treatment of tumor disease or neoplasias, in two or more separate forms of administration, if desired.

37. Drugs according to claim 36, characterized in that the additional active ingredient or ingredients is or are selected from:
Nitrogen derivatives of mustard gas, e.g. cyclophosphamide
Aziridines or epoxides, e.g. thiotepa
Alkyl alkane sulfonates, e.g. busulfan
Nitroso urea compounds, e.g. carmustin
Monofunctional or non-classical alkylating agents, e.g. procarbazine, adozelesine
Platinum derivatives, e.g. cisplatin, carboplatin
Folic acid antagonists or antifolates, e.g. methotrexate
Purine or purine nucleoside analogs, e.g. 6-mercaptopurine, pentostatin
Pyrimidine or pyrimidine nucleoside analogs, e.g. 5-fluorou,racil 5-fluorouridine, 5-fluorodeoxyuridine, capecitabine, tegafur, carmofur, ftorafur
Anthracyclins or chemically related intercalating compounds, e.g. doxorubicin or its morpholino derivatives, MX-2
Antibiotic cytostatic or chemotherapeutic agents, e.g. bleomycin Inhibitors of microtubules (e.g. Vinca alkaloids, taxanes), topoisomerase (e.g. epipodophyllotoxins), phosphatase, tyrosine kinase, thymidylate synthase, DNA or RNA polymerase, histone deacylase, metalloproteinase (e.g. marimastat), protein kinase C (e.g. staurosporine), P glycoprotein, cyclooxygenase-2, adenosine deaminase, farnesyl transferase or angiogenesis
Agonists or inductors of apoptosis (e.g. AOP 99.0001)
Corticoids, e.g. cortisone, prednisone.

38. Drugs according to claim 36, characterized in that the additional active ingredient or ingredients is or are selected from: hormones (e.g. androgens, estrogens, gestagens); antihormones (e.g. antiandrogens, antiestrogens [e.g. tamoxifen, toremifen], antigestagens); inhibitors of the releasing hormones, their analogs, antagonists or superagonists (e.g. buserelin, leuprorelin); aromatase (e.g. aminoglutethimide); or 5a reductase-inhibitors.

39. Drugs according to claim 36, characterized in that the additional active ingredient or ingredients is or are selected from: uracil, 3′-ethinyluridine, 3′-ethinylcytidine, tegafur (1-[2-tetrahydrofuranyl]-5-fluorouracil), fluoropyrimidines, dihydropyrimidine dehydrogenase (DPD) inhibitors (e.g. chloro-2,4-dihydroxy-pyrimidine, 3-cyano-2,6-dihydroxy-pyrimidine, 5-eth inyl2,4(1H,3H)-pyrimidinedione).

40. Drugs according to claim 36, characterized in that the additional active ingredient or ingredients is or are selected from the following dihydropyrimidine dehydrogenase(DPD) inhibitors or inhibitor formulations:
UFT, a combination of uracil and tegafur (1-[2-tetrahydrofuranyl]-5-fluorouracil) at a fixed molar ratio of 4:1;
S-1 (BMS 247617), a combination oftegafur and the two 5-fluorouracil modulators, CDHF(chloro-2,4-dihydroxypyrimidine, a potent DPD inhibitor) and potassium oxonate,
BOF-A2 (emitefur), a drug consisting of 1-ethoxymethyl-5-fluorouracil (EMFU) and 3-cyano-2,6-dihydroxypyridine (CNDP), a potent DPD inhibitor, Eniluracil (5-ethinyl-2,4(1H,3H)-pyrimidinedione), a potent and irreversible DPO inhibitor.
Tegafur (1[2-tetrahydrofuranyl]-5-fluorouracil).

41. Drugs according to claim 36, characterized in that the additional active ingredient or ingredients is or are selected from the cytokines such as for example interleukins, interferons, tumor necrosis factors or transforming growth factors.

42. Drugs according to claim 36, characterized in that the additional active ingredient or ingredients is or are selected from hematopoetic growth factors such as for example erythropoietin, thrombopoietin, granulocyte colonystimulating factor (G-CSF), granulocyte-macrophage colony-stimulating factor (GM-CSF), macrophage colony-stimulating factor (M-CSF).

43. Drugs according claim 34, characterized in that means for active specific immunotherapy (application of irradiated tumor cells, tumor-associated antigens, virus-infected or genetically modified tumor cells [e.g. cytokine gene transfectants]) or for unspecific immunotherapy (e.g. application of immunostimulating or—modulating agents (e.g. BCG, iscador, levamisol, ubenimex, bestatin, Ok-432) or for passive humoral immunotherapy (e.g. application of murine, human, humanized or bispecific monoclonal antibodies [e.g. C225] immunoonjugates [e.g. radioisotope-,cytostatic agent-or toxin-coupled monoclonal antibodies or immunotoxins], immunoglobulin-T-cells chimaeras) or for cellular immunotherapy (e.g. adoptive immunotherapies with cytotoxic effector cells [e.g. lymphokine-activited or natural killer cells, tumor-infiltrating lymphocytes, cytotoxic T-lymphocytes], transfer of genetically modified effector cells [gene therepy]) are combined with compounds corresponding to formula 1.

44. Method for the treatment of tumors, characterized in that a drug according to at least one of the claims 34 is used.

45. Method according to claim 44, whereby the drugs are used in combination with specific or unspecific, active or with humoral or cellular passive modalities of immunotherapy.

46. Method according to claim 45, whereby the specific active immunotherapies are selected from injection or application of irradiated tumor cells or tumorassociated antigens or immunization with genetically modified tumor cells, e.g. with cytokine gene transfectants, or with virus-infected tumor cells.

47. Method according to claim 45, whereby the unspecific active immunotherapies are selected from application of immunostimulating or modulating substances, such as for example BCG, iscador, Ok-432, levamisol, ubenimex, lentinam, bestatin, MER, MTP—PE.

48. Method according to claim 45, whereby the passive humoral immunotherapies are selected from injection or application of murine, human or humanized monoclonal antibodies or immuno-conjugates, e.g. radioisotope-, cytostatic agent- or toxin-coupled (immunotoxins) monoclonal antibodies (e.g. gentuzumab, edrecolomab, trastuzumab, rituximab, lintuzumab, ACA-11, V-10500, Anti-HM1.24 MAB, C225) or geneticallymodified monoclonal antibodies, bispecific antibodies or immunoglobulin-Tcell receptor chimaeras.

49. Method according to claim 45, whereby the passive cellular immunotherapies are selected from adoptive immunotherapies with cytotoxic effector cells, such as for example lymphokine-activated killer cells (LAK), adherent LAK, large granular lymphocytes (LGL), natural killer cells (NK), tumor-infiltrating lymphocytes (TIL), dendritic cells or cytotoxic T-lymphocytes (CTL) as well as the transfer of genetically-modified effector cells (gene therapy, e.g. adenoviral-p53).

50. Method according to claim 44, whereby the drugs are used in combination with radiotherapy.
